(19) United States
(12) Patent Application Publication
(10) Pub. No.: US 2018/0256617 A1
(43) Pub. Date: Sep. 13, 2018

(54) METHODS FOR EXTENDING LIFESPAN AND METHODS OF SCREENING KNOWN PHARMACOLOGICAL AGENTS FOR NEW USES

(71) Applicant: Longevica Therapeutics Inc., Princeton, NJ (US)

(72) Inventors: Alexey G. Ryazanov, Princeton, NJ (US); Alexander V. Chikunov, Princeton, NJ (US); Yuriy Shymkiv, Princeton, NJ (US)

(21) Appl. No.: 15/784,869

(22) Filed: Oct. 16, 2017

Related U.S. Application Data
(63) Continuation of application No. PCT/US16/27859, filed on Apr. 15, 2016.
(60) Provisional application No. 62/148,593, filed on Apr. 16, 2015.

(51) Int. Cl. A61K 31/704; A61K 31/733; A61K 31/198; A61K 45/06; A61P 35/00; A61P 25/28; A61P 3/04; A61P 3/10

(52) U.S. Cl. CPC A61K 31/704; A61K 31/733; A61K 31/198; A61K 45/06; G01N 2500/00; A61P 25/28; A61P 3/04; A61P 3/10; A61K 2300/00; A61P 35/00

(57) ABSTRACT

The present disclosure relates to methods of extending the lifespan of a subject or treating, suppressing, inhibiting or delaying the onset of an age-related condition or disorder, such as cancer, in a subject. The methods comprise administering to the subject an effective amount of (i) a compound that sustains pharmacological activation of xenobiotic metabolism, (ii) a cardiac glycoside, (iii) a chelator, (iv) inulin, (v) D-valine, or any combination thereof. In a further aspect, the present disclosure relates to a method of identifying clinical candidates by performing a high throughput screening in mammals of test compounds which have great structural and/or functional diversity.

This application claims the benefit of U.S. Provisional Application No. 62/148,593, filed Apr. 16, 2015, which is hereby incorporated by reference.

### FIELD OF THE INVENTION

The present invention relates to a method of extending the lifespan, providing anti-aging benefits, and/or treating, or delaying the onset of, an age-related condition or disorder, such as cancer, in a subject by administering one or more compounds that sustain pharmacological activation of xenobiotic metabolism or chelate metal ions in the subject. A further aspect of the invention is a method of identifying clinical candidates by performing a high throughput screening in mammals of test compounds which have great structural and/or functional diversity.

### BACKGROUND OF THE INVENTION

Study of human aging processes is important, in part because many diseases or conditions become more prominent among aged people, for example, cancers, Alzheimer's disease, Parkinson's disease, stroke, heart failure, and heart attack, among which many still lack effective preventive or treatment methods. Aging-associated medical and psychiatric disorders affect multiple systems. Typical disorders of aging also include reduced cardiac output, mood and cognitive changes, muscle wasting, decreased energy, abdominal fat/truncal obesity, weak bones, thin skin and skin wrinkles, and poor sleep.

For example, over time, the heart muscle becomes less efficient, working harder to pump the same amount of blood. In addition, blood vessels lose some of their elasticity and hardened fatty deposits may form on the inner walls of arteries (atherosclerosis). These changes make arteries stiffer, causing the heart to work even harder to pump blood through them. This can lead to high blood pressure (hypertension) and other cardiovascular problems.

With age, bones tend to shrink in size and density, which weakens them and makes them more susceptible to fracture. Muscles generally lose strength and flexibility, and individuals may become less coordinated or have trouble balancing.

Further, memory tends to become less efficient with age, as the number of cells (neurons) in the brain decreases. It may take longer to learn new things or remember familiar words or names.

With age, the eyes are less able to produce tears, the retinas thin, and the lenses gradually become less clear. Focusing on objects that are close up may become more difficult. People may become more sensitive to glare and have trouble adapting to different levels of light. Hearing may dim somewhat as well, in particular hearing high frequencies or following a conversation in a crowded room.

With less saliva to wash away bacteria, teeth and gums become slightly more vulnerable to decay and infection. Teeth also may darken slightly and become more brittle and easier to break.

With age, skin thins and becomes less elastic and more fragile and bruises more easily. Decreased production of natural oils may make skin drier and more wrinkled. Age spots can occur, and small growths called skin tags are more common.

Maintaining a healthy weight or losing weight if overweight is more difficult as one ages. Muscle mass tends to decrease with age, which leads to an increase in fat.

Additional disorders include sarcopenia, diastolic dysfunction, immune deficiencies, and mobility problems. Age-related mobility problems are a very serious issue with far reaching health consequences. Age-related mobility problems lead to an increase in falls, hospitalizations, and future requirements for a caregiver, and increase the risk for depression, osteoporosis, arthritis, congestive heart failure, muscle pain, stroke, dementia and even death.

While treatments exist for some symptoms of age related disorders, no treatments are available that address all aspect of aging simultaneously. There is, therefore a need for new treatment to extend lifespan and treat, or delay the onset of, age-related conditions and disorders.

Xenobiotic metabolism is a complex, highly regulated and energetically costly process whose major function is biotransformation and elimination from the body of lipophilic toxic molecules that are generated as products of metabolism (endobiotics) or absorbed from the environment (xenobiotics). This process involves a large battery of enzymes, mainly expressed in the liver and the gastrointestinal tract and transcriptionally regulated by several nuclear receptors (NRs). Phase I xenobiotic metabolism enzymes, such as cytochrome P450s, catalyze biotransformation reactions (e.g. hydroxylation) to functionalize the chemically inert xenobiotic molecules for further modifications that occur during Phase II. Phase II enzymes (including UDP-glucuronosyltransferases and glutathione transferases) catalyze covalent attachment of polar side groups to functionalized xenobiotic molecules, increasing their solubility and promoting their excretion.

Additionally, drug discovery currently entails finding a potential mechanism by which a disorder or condition may operate, and then disrupting that mechanism to obtain the desired result. The mechanisms of many disorders and conditions, however, have not yet been ascertained or have only been partially determined. Despite decades of researching many conditions such as aging and Alzheimer's disease, the drugs on the market for treating them are still inadequate. There is, therefore, also a need for a more rapid method for discovering effective treatments to disorders and conditions for which adequate therapies have not already been developed and are not well understood.

### SUMMARY OF THE INVENTION

In one aspect, the present invention relates to a method of extending the lifespan of a subject or treating, suppressing, inhibiting, or delaying the onset of, an age-related condition or disorder in a subject. The method comprises administering to the subject an effective amount of (i) a compound that sustains pharmacological activation of xenobiotic metabolism, (ii) a cardiac glycoside, (iii) a chelator (chelating agent), (iv) inulin, (v) agents which induce fermentation by gut bacteria to produce substances that activate xenobiotic metabolism enzymes and/or stimulate xenobiotic excretion (e.g., inulin), (vi) D-valine, or (vii) any combination thereof. The methods described herein can be performed with or without dietary restriction.

In one embodiment, the administration prolongs the lifespan of the subject relative to the lifespan in the absence of the administration. In another embodiment, the administration treats, or delays the onset of, the age-related disease in the subject, relative to the absence of the administration. In yet another embodiment, the administration increases drug metabolism.

In one embodiment, the compound that sustains pharmacological activation of xenobiotic metabolism is an agonist of nuclear receptors that activate xenobiotic metabolism. In a preferred embodiment, the agonist of nuclear receptors that activate xenobiotic metabolism is a constitutive androstane receptor (CAR) agonist, a pregnane X receptor (PXR) agonist, a peroxisome proliferator-activated receptor α (PPARα) agonist, a COX inhibitor, an antiparasitic agent, an acetylcholinesterase inhibitor, an adenosine receptor antagonist, a chelating agent, or any combination thereof.

In one embodiment, any of the methods described herein do not involve administering an estrogen receptor agonist (such as 17β-estradiol, estrone, diethylstilbestrol or hexestrol). In another embodiment, the subject is not being treated with an estrogen receptor agonist. (such as 17β-estradiol, estrone, diethylstilbestrol or hexestrol). In a further embodiment, the methods of treatment described herein further include discontinuing any treatment with an estrogen receptor agonist (such as 17β-estradiol, estrone, diethylstilbestrol or hexestrol).

In one embodiment, the age-related condition or disorder is selected from cancer (e.g., primary and metastatic malignant solid tumor disease and hematological malignancy), neurodegenerative diseases (such as Alzheimer's disease), metabolism disorders (such as diabetes), obesity, or any combination thereof. The methods of the present invention can be used in conjunction with other methods of treating cancer, such as chemotherapy and radiation. Chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, temazolomide (an aqueous form of DTIC), or any analog or derivative variant of the foregoing.

Another embodiment is a method of treatment comprising administering to the subject an effective amount of (i) a compound that sustains pharmacological activation of xenobiotic metabolism, (ii) a cardiac glycoside, (iii) a chelator, (iv) inulin, (v) D-valine, or any combination thereof, to extend the life expectancy (e.g., mean or maximal lifespan) or delay the onset of morbidity of the subject.

Yet another embodiment is a method of decreasing the risk of mortality of a subject by administering to the subject an effective amount of (i) a compound that sustains pharmacological activation of xenobiotic metabolism, (ii) a cardiac glycoside, (iii) a chelator (e.g., an agent that chelates metal ions), (iv) inulin, (v) D-valine, or any combination thereof.

Yet another embodiment is a method of increasing in a subject the amount (e.g., plasma levels or blood levels) of an active compound selected from (i) a compound that sustains pharmacological activation of xenobiotic metabolism, (ii) a cardiac glycoside, (iii) a chelator, (iv) inulin, (v) D-valine, or any combination thereof. The method includes administering to the subject (e.g., once daily, twice daily, or more frequently) for a period of at least 1, 2, 3, 4, 6, 8, 10, 12, 18, or 24 months the active compound (e.g., orally).

Yet another embodiment is a method of supplementing to a subject an active compound selected from (i) a compound that sustains pharmacological activation of xenobiotic metabolism, (ii) a cardiac glycoside, (iii) a chelator, (iv) inulin, (v) D-valine, or any combination thereof. The method includes administering to the subject (e.g., once daily, twice daily, or more frequently) for a period of at least 1, 2, 3, 4, 6, 8, 10, 12, 18, or 24 months the active compound (e.g., orally) an amount that is effective in increasing an initial physiological concentration of active compound in the subject by at least about 10%, wherein the initial physiological concentration is measured from a body fluid sample taken from the subject under a fasting condition (e.g., when the concentration of the active compound is measured after fasting for at least about 12 hours).

In one embodiment, the health benefits of the methods of the present invention mimic those of caloric restriction (i.e., subjects who eat a caloric restricted diet).

In another aspect, the present invention relates to a dietary supplement (e.g., an orally dietary supplement) that contains a compound selected from an agonist of nuclear receptors that activate xenobiotic metabolism, a cardiac glycoside, a chelator, inulin, D-valine, or any combination thereof. The dietary supplement may be used in the methods described herein.

In another embodiment, the dietary supplement further includes one or more vitamins, minerals, fatty acids, antioxidants, amino acids, palatants, nutraceutical additives, or any combination thereof.

In one embodiment, the dietary supplement does not contain an estrogen receptor agonist.

In another aspect, the present invention relates to a method of identifying clinical candidates by performing a high throughput screening in mammals of test compounds which have great structural and/or functional diversity. Each test compound has a known pharmacological or physiological effect, or has drug-like properties (such as hydrophobicity and/or polar groups).

One embodiment of this aspect of the invention is a method of screening for a compound effective for treating a target condition or disorder in a mammal. The method includes:

a) selecting a target condition or disorder,

b) selecting one hundred or more test compounds from each of twenty or more structural and/or functional (e.g., pharmacological and/or physiological) classes of compounds, wherein (i) each of the test compounds has a known pharmacological or physiological effect or drug-like properties (e.g., hydrophilicity and/or inclusion of polar groups), and (ii) none of the selected test compounds are known, at the time of screening, to treat the target condition or disorder, or to substantially effect a biological pathway known to treat the target condition or disorder,

c) selecting a single route of administration and a single dosage form for the test compounds,

d) determining dosing amounts for each test compound at least based on (i) the selected route of administration, and (ii) known toxicity data,

e) optionally,
i) selecting one or more reference compounds for evaluation, wherein the reference compounds are known to treat the target condition or disorder, or to substantially effect a biological pathway known to treat the target condition or disorder,
ii) determining dosing amounts for each reference compound at least based on (i) the selected route of administration, (ii) known toxicity data, and optionally (iii) known efficacy data for the target condition or disorder,

f) performing high-throughput screening with the test compounds in mammals, the screening comprising for each test compound and reference compound,
i) administering the compound to a sufficient number of mammals having the condition or disorder or which is a model for the condition or disorder, such that a statistically significant number of mammals are administered the compound, and
ii) evaluating the target condition or disorder in mammal, and

g) selecting one or more of the test compounds which had positive evaluations for the target condition or disorder.

In one embodiment, a positive evaluation requires at least a 10, 15, 20, or 25% greater effect than observed with a reference compound.

The screening method may further include the step of h) further testing the test compounds which had positive evaluations for the condition or disorder, for example, in different in vitro and/or in vivo assays.

Yet another embodiment is a method of screening for a compound effective for treating a target condition or disorder in a mammal (such as obesity or aging). The method includes:

a) selecting one hundred or more test compounds from each of twenty or more classes of compounds, wherein (i) each of the test compounds has been previously administered to humans and has a known pharmacological effect in humans, and (ii) none of the selected test compounds are known, at the time of screening, to treat the target condition or disorder, or to substantially effect a biological pathway known to treat the target condition or disorder,

b) selecting a single route of administration and a single dosage form for the test compounds,

c) determining dosing amounts for each test compound based on (i) the selected route of administration, (ii) known toxicity data, and optionally (iii) known efficacy data for conditions or disorders it is known to treat,

d) optionally,
i) selecting one or more reference compounds for evaluation, wherein the reference compounds are known to treat the target condition or disorder, or to substantially effect a biological pathway known to treat the target condition or disorder,
ii) determining dosing amounts for each reference compound based on (i) the selected route of administration, (ii) known toxicity data, and (iii) known efficacy data for the target condition or disorder,

e) for each test compound and reference compound,
i) administering the compound to a sufficient number of mammals having the condition or disorder or which is a model for the condition or disorder, according to a dosage regimen over an evaluation period, which is at least one year, such that a statistically significant number of mammals are administered the compound, and
ii) evaluating the condition in the animal one or more times over time, and

f) selecting one or more of the test compounds which had positive evaluations for the condition or disorder.

In one embodiment, at least 100, 200, 400, 500, 600, 800, or 1000 test compounds are selected in step (a) and evaluated.

The evaluation period may be at least 18 months, 2 years, or 2½ years, such as from about 1 year to 5 years, from about 1 year to 4 years, from about 1 year to 3 years or from about 18 months to 3 years.

In one embodiment, a positive evaluation requires at least a 10, 15, 20, or 25% greater effect than observed with a reference compound.

The screening method may further include the step of g) further testing the test compounds which had positive evaluations for the condition or disorder, for example, in different in vitro and/or in vivo assays.

Yet another embodiment is a method of screening for a compound effective for treating a target condition or disorder in a mammal, the method comprising:

a) selecting one hundred or more test compounds from each of twenty or more classes of compounds, wherein (i) each of the test compounds has been previously administered to humans and has a known pharmacological effect in humans, and (ii) none of the selected test compounds are known, at the time of screening, to substantially effect a biological pathway known to treat the target condition or disorder,

b) selecting a single route of administration and a single dosage form for the test compounds,

c) determining dosing amounts for each test compound based on (i) the selected route of administration, (ii) known toxicity data, and (iii) known efficacy data, if available, for conditions or disorders it is known to treat,

d) optionally,
i) selecting one or more reference compounds for evaluation, wherein the reference compounds are known to substantially effect a biological pathway known to treat the target condition or disorder,
ii) determining dosing amounts for each reference compound based on (i) the selected route of administration, (ii) known toxicity data, and (iii) known efficacy data for the target condition or disorder,

e) for each test compound and reference compound,
i) administering the compound to a sufficient number of mammals having the condition or disorder or which is a model for the condition or disorder, such that a statistically significant number of mammals are administered the compound, and
ii) evaluating the condition in the animal one or more times over time, and

f) selecting one or more of the evaluated test compounds which had positive evaluations for the condition or disorder.

The administration of the test compounds and/or observation of the mammals in the screening methods of the present invention can be automated.

In one preferred embodiment, the screening methods of the present invention are used to identify anti-aging agents. For example, each test compound can be administered to a group of mice (e.g., 12, 13, 14, or 15 or more mice) over an extended period of time (for instance, 18 months, 2 years, 2½ years, or 3 years). A test compound may be considered effective if it extends lifespan by 15, 20, 25% or more.

The screening methods of the present invention may also be used to identify anti-obesity agents, agents for treating Alzheimer's disease, and anti-cancer agents.

Yet another embodiment is a method of identifying a new structural class of compounds useful for the prophylaxis or treatment of a target disorder. The method includes the steps of any of the foregoing screening methods. The method may further include the steps of preparing a series of derivatives of a compound identified by the screening method, and testing the derivatives in an in vitro or in vivo model for the target condition or disorder.

In the screening and identification methods described above, drugs with common cellular targets (e.g., common mechanisms of action) may be classed together for determining whether the drug class provides statistically significant results a target condition or disorder (such as obesity, aging, or extending lifespan).

In one embodiment, the methods described herein involve (e.g., for determining extension of lifespan) measuring one or more of: (1) maximum lifespan, (2) mean lifespan, and (3) onset of morbidity (minimum lifespan).

### BRIEF DESCRIPTION OF THE FIGURES

FIG. 1A is a graph showing the survival of male and female B6C3F1/J mice described in Example 1 over their lifespans.

FIG. 1B is a graph showing the survival of the B6C3F1/J mice on a standard diet, purified diet, or caloric restriction as described in Example 1 over their lifespans.

FIG. 2 is a histogram comparing the lifespan of the control and compound-treated B6C3F1/J mice described in Example 1.

FIG. 3 depicts a global view of the screening results described in Example 1, highlighting the effects of individual activators of xenobiotic metabolism.

FIG. 4 depicts the effect of (a) proscillaridin A (b) DTPA, (c) inulin and (d) D-valine on the lifespan of B6C3F1/J mice described in Example 1.

FIG. 5 shows the mean and maximal lifespan data of the B6C3F1/J mice administered the screened compounds, which are characterized by pharmacological use.

### DETAILED DESCRIPTION OF THE INVENTION

The present invention, in part, relates to a method of extending the lifespan of a subject or treating, or delaying the onset of, an age-related condition or disorder in a subject. In one embodiment, the methods described herein comprise administering one or more compounds that sustain pharmacological activation of xenobiotic metabolism in the subject.

### Definitions

The phrase “extending the lifespan” includes statistically significantly increasing the life expectancy of a subject (e.g., compared to a control group).

“Therapeutically effective amount” refers to that amount of a compound of the invention that, when administered to a mammal, is sufficient to effect treatment, as defined below, of a disease or condition in the mammal. The amount of a compound of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest, e.g., tissue injury, in a mammal, having the disease or condition of interest, and includes: (i) preventing the disease or condition from occurring in a mammal, in particular, when such mammal is predisposed to the condition but has not yet been diagnosed as having it; (ii) inhibiting the disease or condition, i.e., arresting its development; (iii) relieving the disease or condition, i.e., causing regression of the disease or condition; or (iv) relieving the symptoms resulting from the disease or condition. As used herein, the terms “disease,” “disorder,” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

### Active Compounds

Xenobiotic metabolism activator compounds useful in the methods described herein, include, for example, compounds that act as agonists of nuclear receptors that activate or induce xenobiotic metabolism.

Suitable classes of agonist compounds include, but are not limited to, constitutive androstane receptor (CAR) agonists, pregnane X receptor (PXR) agonists and peroxisome proliferator-activated receptor α (PPARα) agonists, and any combination thereof.

Suitable examples of CAR agonists include, but are not limited to 6,7-dimethylesculetin, acetaminophen, artemisinin, atorvastatin, cerivastatin, CITCO ((6-(4-chlorophenyl)imidazo[2,1-b][1,3]thiazole-5-carbaldehyde-O-(3,4-dichlorobenzyl)oxime), fluvastatin, orphenadrine, phenobarbital, phenytoin, pravastatin, simvastatin, and combinations thereof.

Suitable examples of PXR agonists include, but are not limited to, 4-hydroxytamoxifen, androstenol, artemisinin, avasimibe, BK8644, bosentan, bromopropylate, butamifos, carbamazepine, cis-guggulsterone, clotrimazole, desmethoxyyangonin, dexamethasone, dihydromethysticin, dymuron, efavirenz, esprocarb, ethion, etoposide, flucythrinate, forskolin, hyperforin (hypericum perforatum extract or St John's wort), indanofan, isofenphos, isradipine, kava extract, lithocholic acid, lovastatin, meclizine, methadone, metlachlor, mevastatin, mifepristone, nicardipine, nifedipine, paclitaxel, PCN (pregnenolone-16α-carbonitrile), phenobarbital, piperophos, pretilachlor, pyributicarb, rifampicin, ritonavir, spironolactone, SR 12813, tamoxifen, thenylchlor, topiramate, topotecan, trans-guggulsteone, triadimefon, ursodeoxycholic acid, and combinations thereof.

Suitable examples of PPARα agonists include, but are not limited to, aleglitazar, arachidonic acid, bezafibrate, ciprofibrate, clinofibrate, clofibrate, clofibric acid, clofibride, CP 775146, eicosapentaenoic acid, etofibrate, fenofibrate, fenofibric acid, gemfibrozil, GW 7647, linoleic acid, muraglitazar, nafenopin, oleic acid, oleylethanolamide, palmitic acid, palmitoleic acid, palmitoylethanolamide, pioglitazone, pirinixic acid, rivoglitazone, ronifibrate, rosiglitazone, saroglitazar, simfibrate, stearic acid, tesaglitazar, troglitazone, WY-14643, and combinations thereof.

Suitable examples of cardiac glycosides include, but are not limited to, acetyldigitoxin, acetyldigoxin, arenobufagin, bufotalin, cinobufagin, cymarin, deslanoside, Digitalis leaves, digitoxin, digoxin, gitoformate, K-strophanthin, lanatoside C, marinobufagin, metildigoxin, ouabain, peruvoside, proscillaridin (e.g., proscillaridin A), scilliroside, and combinations thereof.

Suitable examples of COX inhibitors include, but are not limited to, aspirin, non-steroidal anti-inflammatory drugs (NSAIDs) (such as ibuprofen, piroxicam, mefenamic acid, diclofenac, flurbiprofen, and indomethacin), COX-2 inhibitors (such as celecoxib), and combinations thereof.

Suitable examples of antiparasitic agents include, but are not limited to, mebendazole, pyrantel pamoate, thiabendazole, diethylcarbamazine, ivermectin, niclosamide, praziquantel, albendazole, praziquantel, rifampin, amphotericin B, melarsoprol, eflornithine, metronidazole, tinidazole, miltefosine, and combinations thereof.

Suitable examples of acetylcholinesterase inhibitors include, but are not limited to, donepezil, galantamine, caffeine, delta9-tetrahydrocannabinol (THC), physostigmine, neostigmine, pyridostigmine, ambenonium, demecarium, rivastigmine, tacrine, edrophonium, huperzine A, ladostigil, ungeremine, lactucopicrin, and combinations thereof.

Suitable examples of adenosine receptor antagonists include, but are not limited to, caffeine, 8-cyclopentyl-1,3-dipropylxanthine (DPCPX), cyclopentyltheophylline (CPT), istradefylline, SCH-58261, dyphylline, theophylline, theobromine, proxyphylline, pentoxiphylline, etofylline, aminophylline, dimenhydrinate, and combinations thereof.

Suitable examples of chelators include, but are not limited to, 2,3-dimercapto-1-propanesulfonic acid (DMPS), alpha lipoic acid, BAPTA, citric acid, deferasirox, deferiprone, deferoxamine (DFO), dimercaprol (BAL), dimercaptosuccinic acid (DMSA), ethylene glycol tetraacetic acid (EGTA), ethylenediaminetetraacetic acid (EDTA), penicillamine, pentetic acid (DTPA), Prussian blue, succinic acid, tartaric acid, triethylenetetramine (TETA), N-acetyl-L-cysteine, aspartic acid, histidine, glutamic acid, glutathione, etindronic acid, methionine, selenomethionine, taurine, alendronic acid, clodronic acid, tiopronin, diethyldithiocarbamate, and combinations thereof.

Additional inducers of xenobiotic metabolism include, but are not limited to, 2-naphthoflavone, 3-methylcholanthrene, dioxin, metyrapone, decitabine, trichostatin A, hydroxymethylpyrene, indolo[3,2-b]carbazole, phenethyl isothiocyanate, isothiocyanatomethylbenzene, sulforaphane, coumestrol, testosterone, dihydrotestosterone, indole-3-carbinol, 3-nitrobenzanthrone, 2-(4-amino-3-methylphenyl)-5-fluorobenzothiazole, primaquine, iprodione, ketoconazole, deltamethrin, omeprazole, pentachlorophenol, fipronil, sulindac, 3-aminobenzanthrone, 6-nitrochrysene, Itraconazole, Enilconazole (imazalil), 2-[2-(acetylamino)-4-(diallylamino)-5-methoxyphenyl]-5-amino-7-bromo-4-chloro-2H-benzotriazole (PTBA-8), 9-hydroxy-5,6-dimethyl-N-[2-(dimethylamino)ethyl)-6H-pyrido(4,3-b)-carbazole-1-carboxamide, carbaryl, 6-formylindolo[3,2-b]carbazole, 2-[2-(acetylamino)-4-(diethylamino)-5-methoxyphenyl]-5-amino-7-bromo-4-chloro-2H-benzotriazole (PBTA-7), chlorpyrifos, sulindac sulfone, fluconazole, permethrin, ascorbigen, DEET (N,N-diethyl-meta-toluamide), clevidipine, emodin, 4-biphenylamine, nevirapine, efavirenz, chlorpyrifos oxon, ibrolipim, isoniazid, ethanol, tretinoin, fandosentan potassium, modafinil, trazodone, trimeprazine, etoposide, doxorubicin, rifabutin, alpha-naphthoflavone, progesterone, ethambutol, benzil, 1-methylphenanthrene, olopatadine, ethionamide, quercetin, 4-hydroxynonenal, oltipraz, butanoate, resveratrol, paraquat, alpha lipoic acid, carnosic acid, carnosol, oxaliplatin, paclitaxel, nicotinamide, azathioprine, eugenol, chlorophyllin, 2-tert-butylhydroquinone, hydrocortisone, procainamide, corticosterone, medroxyprogesterone 17-acetate, dopamine, 4-nitrophenol, bilirubin, glycyrrhizic acid, naringenin, saccharolactone, isopropyl thiogalactoside, naphthyl glucuronide, epigallocatechin gallate, imipramine, serotonin, nicotine, cotinine, propylpyrazoletriol, genistein, pyrazole, ITE (2-(1H-indol-3-ylcarbonyl)-4-thiazolecarboxylic acid methyl ester), MeBIO ((2′Z,3′E)-6-bromo-1-methylindirubin-3′-oxime), pifithrin-α, and combinations thereof.

### Routes of Administration

Any route of administration may be selected for use in the methods described herein. For instance, the route of administration may be selected from oral, nasal, buccal, rectal, vaginal, ophthalmic, subcutaneous, intramuscular, intraperitoneal, intravenous, intraarterial, intratumoral, spinal, intrathecal, intra-articular, intra-arterial, sub-arachnoid, sublingual, oral mucosal, pulmonary, bronchial, lymphatic, intra-uterine, subcutaneous, intratumor, integrated on an implantable device, intradural, intracortical, dermal, epidermal, transdermal, vaginal, rectal, ocular (for examples through the conjunctiva), intraocular, uretal, and parenteral. A preferred route of administration is oral.

### Dosages

The actual dosage amount of the active compound(s) administered to a subject may be determined by physical and physiological factors such as age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active compound(s) in a composition and appropriate dose(s) for the individual subject.

In one embodiment, a human subject is administered the daily dose of from about 0.01 mg/kg to about 1000 mg/kg of the active compound(s). Preferably, the dosage level will be about 0.1 to about 250 mg/kg per day, more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be about 0.05 to 0.5, about 0.5 to 5 or about 5 to 50 mg/kg per day. For oral administration, the compositions are preferably provided in the form of tablets containing 0.1 to 1000 mg of the active compound(s), for example, about 0.1, 0.5, 1, 5, 10, 15, 20, 25, 50, 75, 100, 125, 150, 175, 200, 225, 250, 275, 300, 350, 400, 450, 500, 550, 600, 650, 700, 750, 800, 850, 900, 950 or about 1000 mg of the active compound(s).

The compounds may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day (once daily), every two days, every three days, every four days, every five days, every six days, a weekly basis, a monthly basis or any set number of days or weeks there-between.

In certain embodiments, the compounds described herein can be administered over an extended period of time, for example, for at least 1, at least 2, at least 6, at least 12, at least 18 or at least 24 months (or, for example, for 18 months, 2 years, 2½ years, or for 3 years).

In one embodiment, the methods described herein do not involve co-administration of estrogen receptor agonists (including endogenous estrogens, such as 17β-estradiol and estrone, and synthetic estrogens, such as diethylstilbestrol and hexestrol).

### Pharmaceutical Compositions

The compounds described herein may be administered (e.g., orally) in the form of a solid or liquid dosage form. In both, the compounds may be coated in a material to protect them from the action of acids and other natural conditions which may inactivate the compounds. The compounds may be formulated as aqueous solutions, liquid dispersions, (ingestible) tablets, buccal tablets, troches, capsules, elixirs, powders, granules, ointments, adhesive skin patches, sprays, suspensions, syrups, and wafers. The dosage forms may include pharmaceutically acceptable excipients, diluents, and/or carriers known in the art, such as binders, disintegrating agents, emulsifiers, lubricants, flavorants, antioxidants, and preservatives. Liquid dosage forms may include diluents such as saline or an aqueous buffer.

The active compounds can be administered as foods, food additives, edible soluble films, drinks, medicinal agents, and feeds for domestic and wild animals. The drinks may be non-alcohol drinks or alcohol drinks. Examples of non-alcohol drinks include carbonated drinks, non-carbonated drinks (such as fruit juice, and nectar), soft drinks, sports drinks, tea, coffee, and hot chocolate. The alcohol drinks may be in the form of, for example, beer, low-malt beer, third-category beer, sake, umeshu, wine, champagne, liqueur, chuhai, or medicated liquor.

For use as a food material or food additive (e.g., human food or animal food, such as dog or cat food or feed for poultry, cows, or pigs), the active compound may be in the form of, for example, a tablet, a capsule formulation, a solid agent (such as a powder and a granule) dissolved in drinks, a semi-solid such as jelly, a liquid (such as drinking water), and a high-concentration solution diluted before use. Optional components, such as vitamins, carbohydrates, dyes, and flavoring agents commonly added to food may be appropriately mixed. The food may be given in any form, including a liquid and a solid.

### Dietary Supplements

In one embodiment, the compounds described herein are administered as part of a dietary supplement (i.e., a dietary supplement formulation). Thus, in a further aspect, the present invention relates a dietary supplement comprising one or more of the compounds described herein, i.e., a compound that sustains pharmacological activation of xenobiotic metabolism (e.g., a CAR agonist, a PXR agonist, or a PPARα agonist), St. John's wort extract or hyperforin, a cardiac glycoside, a chelator, inulin, sinistrin, D-valine, or any combination thereof. In one embodiment, the dietary supplement includes at last two, three, four, or five of the active compounds described herein.

In one embodiment, the present invention relates a dietary supplement comprising (a) an agonist of one or more nuclear receptors that activate xenobiotic metabolism (such as a CAR agonist, a PXR agonist, a PPARα agonist, or any combination thereof) and (b) a compound selected from a cardiac glycoside, a chelator, inulin, D-valine, or any combination thereof.

In additional embodiments, the present invention relates to a dietary supplement comprising at least 1, such as at least 2, as at least 3, as at least 4, as at least 5, as at least 6, as at least 7, as at least 8, as at least 9, as at least 10, as at least 11, as at least 12, as at least 13, as at least 14, as at least 15, as at least 16, as at least 17, as at least 18, as at least 19 or as at least 20 compounds selected from CAR agonists, PXR agonists, PPARα agonists, cardiac glycosides, chelators, inulin, D-valine, and any combination thereof.

The dietary supplement may also comprise one or more compounds known to those of ordinary skill to be useful in dietary supplements, including, but not limited to, vitamins, minerals (e.g., magnesium), fatty acids, antioxidants, amino acids, palatants and nutraceutical additives, and any combination thereof. See the National Institute of Health Dietary Supplement Database: http://www.dsld.nlm.nih.gov/dsld. For example, the dietary supplement may also include one or more calcium-containing materials such as calcium carbonate, stannol esters, hydroxycitric acid, vitamins, minerals, herbals, spices and mixtures thereof. Examples of vitamins that are available as additional ingredients include, but are not limited to, vitamin A (retinol), vitamin D (cholecalciferol), vitamin E group (alpha-tocopherol and other tocopherols), vitamin K group (phylloquinones and menaquinones), thiamine (vitamin B₁), riboflavin (vitamin B₂), niacin, vitamin B₆ group, folic acid, vitamin B₁₂ (cobalamins), biotin, vitamin C (ascorbic acid), and mixtures thereof. The amount of vitamin or vitamins present in the final product is dependent on the particular vitamin. Examples of minerals that are available as additional ingredients include, but are not limited to, calcium, magnesium, phosphorus, iron, zinc, iodine, selenium, potassium, copper, manganese, molybdenum and mixtures thereof. As is the case with vitamins, the amount of mineral or minerals present in the dietary supplement is dependent on the particular mineral.

In one embodiment, the dietary supplement described herein does not contain, or include instructions not to co-administer estrogen receptor agonists (including endogenous estrogens, such as 17β-estradiol and estrone, and synthetic estrogens, such as diethylstilbestrol and hexestrol).

### Subject

The subject is a mammal, such as a human, or, for example, a domestic or wild animal, such as a chicken, quail, ostrich, horse, bird, dog, cat, cow or pig. Preferably, the subject is a human, such as a male or female adult. For example, the subject may be an adult who is between 18 and 30 years old, between 30 and 40 years old, between 40 and 50 years old, between 50 and 60 years old, between 60 and 70 years old, and between 70 and 80 years old.

### Screening Methods

Target Condition or Disorder

The target condition or disorder may be one in which no target or biochemical pathway is known, and/or there is only a partial understanding as to the cause of the condition or disorder. The target condition or disorder may be one for which known treatments are inadequate. For instance, the target condition or disorder may be aging, obesity, Alzheimer's disease, or cancer. The screening methods may also be used to assess mortality.

Screening Test Compounds

The test compounds have great structural and/or functional (e.g., pharmacological or physiological) diversity. For instance, test compounds may be selected from 40, 50, 60, 70, 80, 90, 100, 120, 150, 200, 300, 400, 500, 600, 700, 800, 900, or 1000 or more structural and/or functional classes of compounds.

In one embodiment, 200, 300, 400, 500, 600, 700, 800, 900, 1000, 2000, 4000, 5000, 6000, 8000, 9000, 10000 or more test compounds are selected for use in the screening.

The test compounds may have a known pharmacological or physiological effect, for example, in humans. In one embodiment, at least 80 or 90% (e.g., all) of the test compounds have a known pharmacological or physiological effect. In another embodiment, at least 80 or 90% (e.g., all) of the compounds selected in step (b) have a known pharmacological effect in humans.

The test compounds may not have a known pharmacological or physiological effect, but may have drug-like properties, such as hydrophobicity and/or polar groups. For example, the compound may follow the Lipinki rule of five. Such a compound is more likely to be membrane permeable and easily absorbed by the body if it matches the following criteria: (1) its molecular weight is less than 500, (2) the compound's lipophilicity, expressed as a quantity known as log P (the logarithm of the partition coefficient between water and 1-octanol), is less than 5, (3) the number of groups in the molecule that can donate hydrogen atoms to hydrogen bonds (usually the sum of hydroxyl and amine groups in a drug molecule) is less than 5, and (4) the number of groups that can accept hydrogen atoms to form hydrogen bonds (estimated by the sum of oxygen and nitrogen atoms) is less than 10.

The test compounds may have been previously administered to humans (for example, in prior clinical trials for a condition or disorder different from the target condition or disorder). In one embodiment, at least 80 or 90% of the test compounds have been previously administered to humans (e.g., at least 10, 20, 50, 100, 200, or 400 humans). For instance, some or all of the test compounds could have previously been the subject of a human clinical trial (e.g., an uncompleted or completed human clinical trial), or was previously approved by at least one governmental agency for use as a pharmaceutical.

In another embodiment, at least 80 or 90% of the test compounds are active pharmaceutical ingredients previously approved by a governmental regulatory agency (such as the U.S. Food and Drug Administration (FDA) or the European Medicines Agency (EMA)) for prophylaxis or treatment in humans.

Routes of Administration

Any route of administration may be selected for use in the screening. For instance, the route of administration may be selected from oral, nasal, buccal, rectal, vaginal, ophthalmic, subcutaneous, intramuscular, intraperitoneal, intravenous, intraarterial, intratumoral, spinal, intrathecal, intra-articular, intra-arterial, sub-arachnoid, sublingual, oral mucosal, pulmonary, bronchial, lymphatic, intra-uterine, subcutaneous, intratumor, integrated on an implantable device, intradural, intracortical, dermal, epidermal, transdermal, vaginal, rectal, ocular (for examples through the conjunctiva), intraocular, uretal, and parenteral. In one preferred embodiment, the route of administration selected is the oral route.

Dosage Form

Any suitable dosage form may be selected for use in the screening. For instance, the dosage form may be selected from solutions (e.g., oral solutions and intravenous solutions), capsules, tablets, pills, dermal gel, lotion, cream, patch, rectal suppository, gel, and infusions.

In one preferred embodiment, the dosage form is a solution, such as an oral solution. In another embodiment, the dosage form is a capsule.

Dosage Amount

The dosage amount may be determined based on the route of administration as well as one or more of (1) the known LD₅₀ in the mammal, and (2) the maximum recommended therapeutic dose in humans.

For example, if the screening is to be performed in mice, the dosage amount may be 7 to 10 times the maximum recommended therapeutic dose in humans.

In one embodiment, the dosing amounts for one or more test compounds are above that currently approved by a governmental regulatory agency. In another embodiment, the dosing amount for each previously approved test compound is above that currently approved by a governmental regulatory agency for the test compound.

In yet another embodiment, the dosing amount for one or more test compounds are below that currently approved by a governmental regulatory agency. In another embodiment, the dosing amount for each previously approved test compound is below that currently approved by a governmental regulatory agency for the test compound.

Mammals

Any suitable mammal may be used for the screening methods described herein. Suitable mammals include, but are not limited to, rats, mice, dogs, monkeys, pigs, hamsters, and humans. In one preferred embodiment, the mammals used for the screening are mice. In another preferred embodiment, the mammals used for the screening are rats.

In one embodiment, each test compound is administered to at least 5 mammals. In another embodiment, each test compound is administered to at least 6 mammals. In yet another embodiment, each test compound is administered to 6, 7, or 8 mammals. In yet another embodiment, each test compound is administered to at least 12 or 15 mammals. For instance, each test compound can be administered to 10, 11, 12, 13, 14, or 15 mammals (e.g., mice or rats). In one embodiment, each test compound is administered to at least 100 mammals (e.g., rats or hamsters).

Post-Screening

The methods of the present invention can be used to identify chemical classes of compounds which were not previously known to be effective for the treatment of the target condition or disorder. After one or more compounds have been identified as active against a condition or disorder, they may be derivatized to find other more active and/or less toxic compounds suitable for preventing, inhibiting, or treating the target condition or disorder. In one embodiment, a series of derivatives of an identified compound is made to determine the structure-activity relationship for the series. Experiments can also be conducted to determine the pharmacophore of the identified compound, which can help in designing improved compounds for preventing, inhibiting, or treating the target condition or disorder.

### EXAMPLES

### Example 1

Animal Care and Handling:

Long-lived B6C3F1/J mice were produced by one round of breeding of female C57BL/6J and male C3H/HeJ strains and housed at a laboratory in Sacramento, Calif. Compound dosing was initiated at approx. 5 months of age and continued throughout the lifespan of the mice. Longevity data were collected as an endpoint and the welfare of animals was monitored throughout the study. Clinical observations, body weight measurements, taste aversion and water consumption were assessed before the start of dosing and at 6, 12, and 24 months, and, for a subset of mice, before euthanasia. Individual mice were regularly checked for parameters indicating overall health, e.g. hydration level, responsiveness, etc.

Compounds and Dosing:

1035 commercially available chemical compounds were provided to the mice via drinking water. Each compound was purchased from Sigma-Aldrich, weighed, uniquely labeled, and continuously shipped throughout the experiment from a separate site in order to establish a blind experiment. Compounds were administered to the mice at a concentration between 50% and 100% of the maximum recommended therapeutic dose in humans (MRTD), adjusted for mouse dosing. For compounds with unknown MRTD, doses were selected to range between 0.01 and 0.1 of the compound's oral LD₅₀ in the mouse.

Data Analysis All compounds were categorized according to their known pharmacological classification and their biological mechanism of action. 834 of the 1035 compounds tested (81%) placed into at least one category. Mean lifespan values were calculated for individual compounds and for compound groups. A matrix laboratory (MATLAB) computing environment and programming language was used to perform statistical comparisons between controls and dosed mice and between different drug classes. A false discovery rate (FDR) adjusted p-value was calculated for each drug class based on its mean lifespan, the number of compounds it contained and the total number of drug classes under consideration.

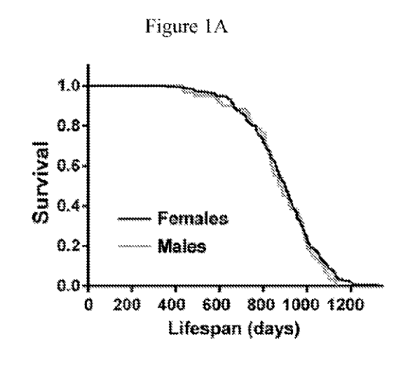

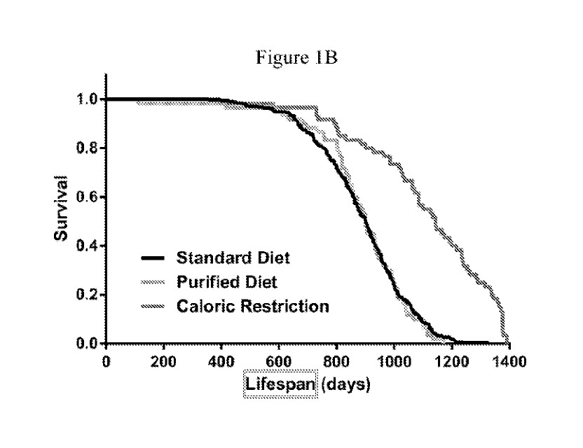

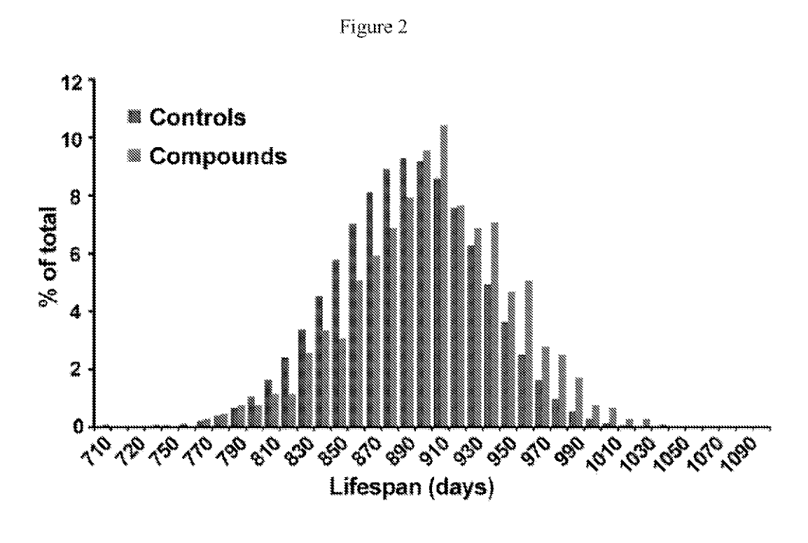

FIG. 1A shows the effect of gender on the lifespan of B6C3F1/J mice. FIG. 1B shows the effect of diet on the lifespan of B6C3F1/J mice. As can be seen from FIG. 1A, the survival curves for female and male mice were nearly identical. Additionally, as shown in FIG. 1B, feeding the mice a purified, chemically defined diet did not alter their lifespan compared to a standard multigrain based diet formulated following the NIH-31 diet specifications. Mouse lifespan was increased (24%) by caloric restriction (60% of ad libitum food intake).

As can be seen from FIG. 2, compound dosing had a generally positive effect on longevity, increasing the mean lifespan of the mice from 866 days for controls to 899 days for the compound-dosed mice. The maximum lifespan (mean lifespan of the most long-lived 10% of the mice) was also extended from 1149 days for the control mice to 1184 days for the compound-dosed mice.

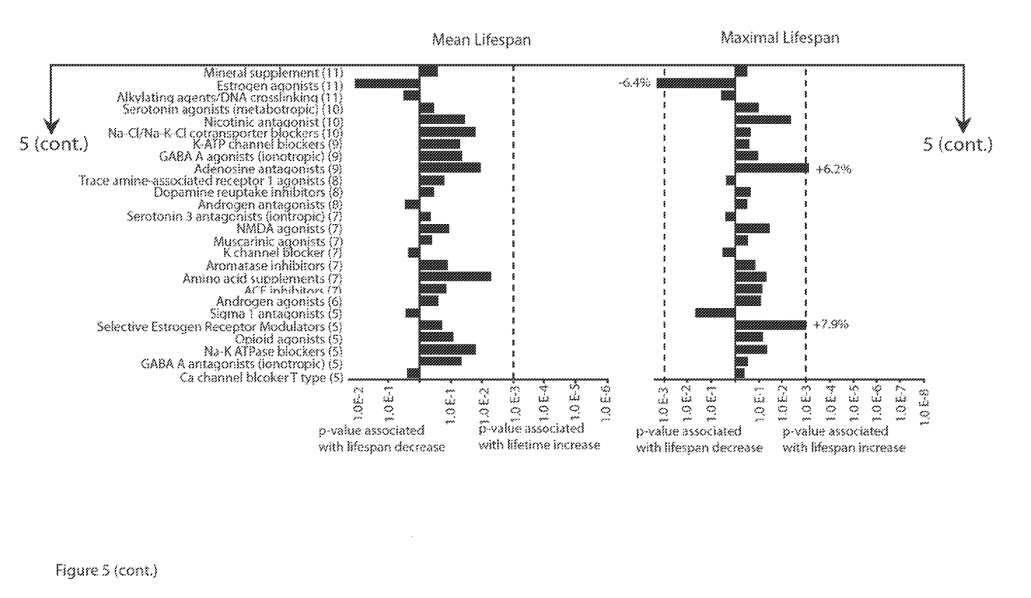

FIG. 5 show lifespan data for the screened compounds, characterized by molecular mechanism of action or cellular target.

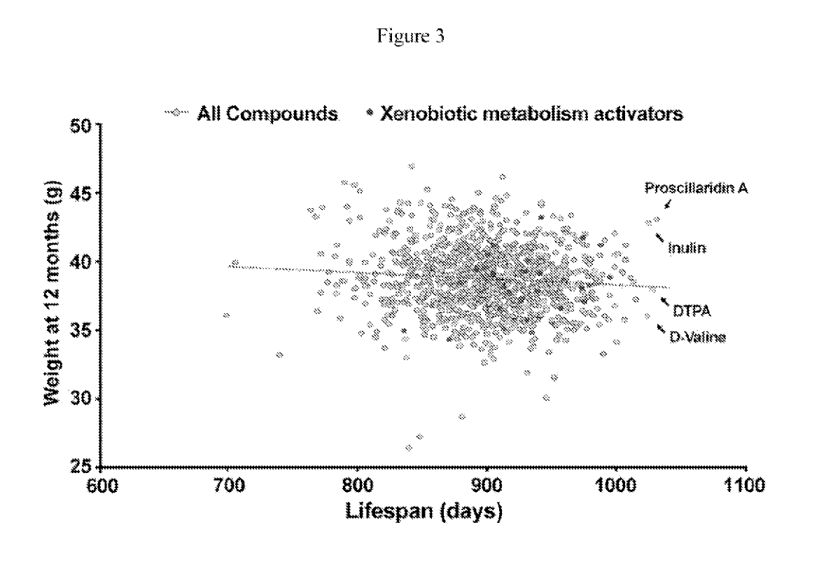

Among all drug classes, activators of xenobiotic metabolism (particularly CAR agonists, PXR agonists and PPARα agonists) exhibited the most significant effect on mouse lifespan, extending lifespan relative not only to control mice, but also to all compound-treated mice. This class of drugs extended the maximal lifespan of the mice to 1216 days. Additionally, the lifespan-extending effects of the CAR agonists, PXR agonists and PPARα agonists are not due to voluntary caloric restriction, because mice dosed with these compounds showed no alterations in body weight relative to the control mice or to all compound-dosed mice. See FIG. 3.

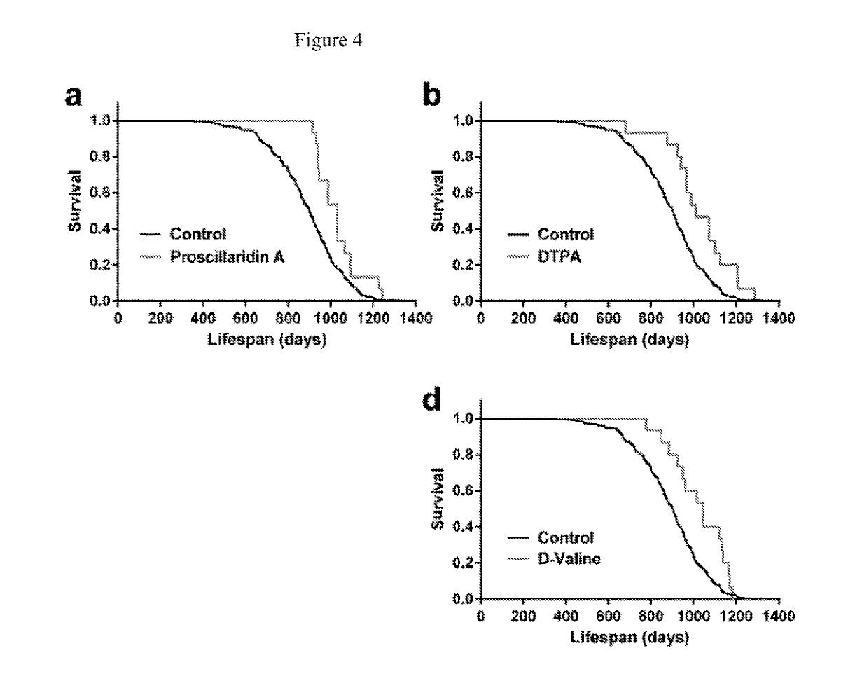

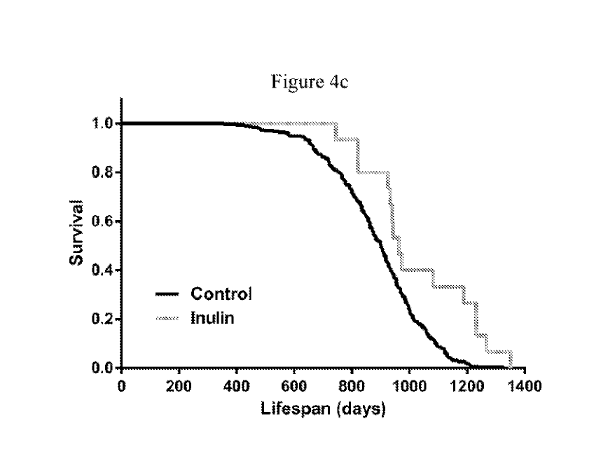

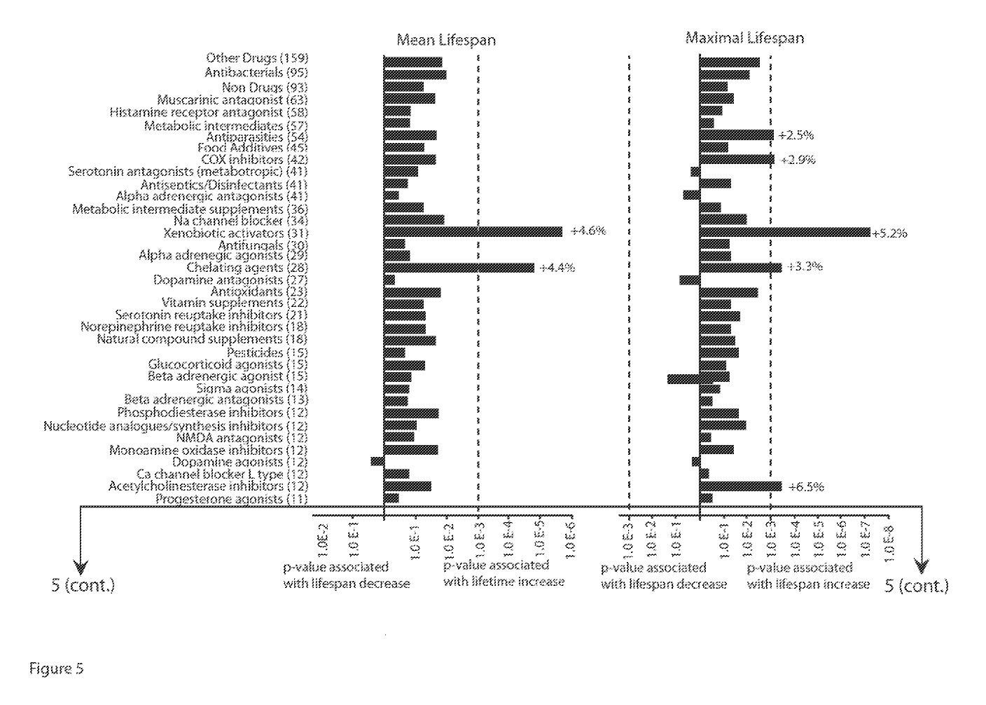

As can also be seen from FIGS. 3 and 4, four individual compounds (the cardiac glycoside proscillaridin A, the chelator DTPA, inulin and D-valine) each increased mouse lifespan by over 15% relative to controls (0.05<p<0.06). Inulin, a soluble fiber, is a prebiotic that affects the gut microbiome composition, promoting the growth of bacteria that produce substances that activate Phase II enzymes and stimulate xenobiotic excretion.

All patents, patent applications, and publications cited herein are incorporated by reference in their entireties.

1. A method of extending the lifespan of a subject or treating, or delaying the onset of, an age-related condition or disorder in a subject, the method comprising administering to the subject an effective amount of a compound selected from the group consisting of (i) a compound that sustains pharmacological activation of xenobiotic metabolism, (ii) a cardiac glycoside, (iii) a chelator, (iv) inulin, (v) D-valine, and any combination thereof.

2. The method of claim 1, wherein the administration prolongs the lifespan of the subject relative to the lifespan in the absence of the administration.

3. The method of claim 1, wherein the administration treating, or delaying the onset of, the age-related disease in the subject, relative to the absence of the administration.

4. The method of claim 1, wherein the compound that sustains pharmacological activation of xenobiotic metabolism is an agonist of nuclear receptors that activate xenobiotic metabolism.

5. The method of claim 4, wherein the agonist of nuclear receptors that activate xenobiotic metabolism is selected from the group consisting of constitutive androstane receptor (CAR) agonists, pregnane X receptor (PXR) agonists, peroxisome proliferator-activated receptor α (PPARα) agonists, chelating agents, COX inhibitors, antiparasitic agents, acetylcholinesterase inhibitors, adenosine receptor antagonists, and any combination thereof.

6. The method of claim 5, wherein the CAR agonist is selected from the group consisting of 6,7-dimethylesculetin, acetaminophen, artemisinin, atorvastatin, cerivastatin, CITCO ((6-(4-chlorophenyl)imidazo[2,1-b][1,3]thiazole-5-carbaldehyde-O-(3,4-dichlorobenzyl)oxime), fluvastatin, orphenadrine, phenobarbital, phenytoin, pravastatin, simvastatin, and any combination thereof.

7. The method of claim 5, wherein the PXR agonist is selected from the group consisting of 4-hydroxytamoxifen, androstenol, artemisinin, avasimibe, BK8644, bosentan, bromopropylate, butamifos, carbamazepine, cis-guggulsterone, clotrimazole, desmethoxyyangonin, dexamethasone, dihydromethysticin, dymuron, efavirenz, esprocarb, ethion, etoposide, flucythrinate, forskolin, hyperforin (hypericum perforatum extract or St John's wort), indanofan, isofenphos, isradipine, kava extract, lithocholic acid, lovastatin, meclizine, methadone, metlachlor, mevastatin, mifepristone, nicardipine, nifedipine, paclitaxel, PCN (pregnenolone-16α-carbonitrile), phenobarbital, piperophos, pretilachlor, pyributicarb, rifampicin, ritonavir, spironolactone, SR 12813, tamoxifen, thenylchlor, topiramate, topotecan, trans-guggulsteone, triadimefon, ursodeoxycholic acid, and any combination thereof.

8. The method of claim 5, wherein the PPARα agonist is selected from the group consisting of aleglitazar, arachidonic acid, bezafibrate, ciprofibrate, clinofibrate, clofibrate, clofibric acid, clofibride, CP 775146, eicosapentaenoic acid, etofibrate, fenofibrate, fenofibric acid, gemfibrozil, GW 7647, linoleic acid, muraglitazar, nafenopin, oleic acid, oleylethanolamide, palmitic acid, palmitoleic acid, palmitoylethanolamide, pioglitazone, pirinixic acid, rivoglitazone, ronifibrate, rosiglitazone, saroglitazar, simfibrate, stearic acid, tesaglitazar, troglitazone, WY-14643, and any combination thereof.

9. The method of claim 1, wherein the cardiac glucoside is selected from the group consisting of acetyldigitoxin, acetyldigoxin, arenobufagin, bufotalin, cinobufagin, cymarin, deslanoside, Digitalis leaves, digitoxin, digoxin, gitoformate, k-strophanthin, lanatoside C, marinobufagin, metildigoxin, ouabain, peruvoside, proscillaridin, scilliroside, and any combination thereof.

10. The method of claim 1, wherein the chelator is selected from 2,3-dimercapto-1-propanesulfonic acid (DMPS), alpha lipoic acid, BAPTA, citric acid, deferasirox, deferiprone, deferoxamine (DFO), dimercaprol (BAL), dimercaptosuccinic acid (DMSA), ethylene glycol tetraacetic acid (EGTA), ethylenediaminetetraacetic acid (EDTA), penicillamine, pentetic acid (DTPA), Prussian blue, succinic acid, tartaric acid, triethylenetetramine (TETA), and any combination thereof.

11. The method of claim 1, wherein the compound that sustains pharmacological activation of xenobiotic metabolism is selected from the group consisting of 2-naphthoflavone, 3-methylcholanthrene, dioxin, metyrapone, decitabine, trichostatin A, hydroxymethylpyrene, indolo[3,2-b]carbazole, phenethyl isothiocyanate, isothiocyanatomethylbenzene, sulforaphane, coumestrol, testosterone, dihydrotestosterone, indole-3-carbinol, 3-nitrobenzanthrone, 2-(4-amino-3-methylphenyl)-5-fluorobenzothiazole, primaquine, iprodione, ketoconazole, deltamethrin, omeprazole, pentachlorophenol, fipronil, sulindac, 3-aminobenzanthrone, 6-nitrochrysene, itraconazole, enilconazole (imazalil), 2-[2-(acetylamino)-4-(diallylamino)-5-methoxyphenyl]-5-amino-7-bromo-4-chloro-2H-benzotriazole (PTBA-8), 9-hydroxy-5,6-dimethyl-N-[2-(dimethylamino)ethyl)-6H-pyrido(4,3-b)-carbazole-1-carboxamide, carbaryl, 6-formylindolo[3,2-b]carbazole, 2-[2-(acetylamino)-4-(diethylamino)-5-methoxyphenyl]-5-amino-7-bromo-4-chloro-2H-benzotriazole (PBTA-7), chlorpyrifos, sulindac sulfone, fluconazole, permethrin, ascorbigen, DEET (N,N-diethyl-meta-toluamide), clevidipine, emodin, 4-biphenylamine, nevirapine, efavirenz, chlorpyrifos oxon, ibrolipim, isoniazid, ethanol, tretinoin, fandosentan potassium, modafinil, trazodone, trimeprazine, etoposide, doxorubicin, rifabutin, alpha-naphthoflavone, progesterone, ethambutol, benzil, 1-methylphenanthrene, olopatadine, ethionamide, quercetin, 4-hydroxynonenal, oltipraz, butanoate, resveratrol, paraquat, alpha lipoic acid, carnosic acid, carnosol, oxaliplatin, paclitaxel, nicotinamide, azathioprine, eugenol, chlorophyllin, 2-tert-butylhydroquinone, hydrocortisone, procainamide, corticosterone, medroxyprogesterone 17-acetate, dopamine, 4-nitrophenol, bilirubin, glycyrrhizic acid, naringenin, saccharolactone, isopropyl thiogalactoside, naphthyl glucuronide, epigallocatechin gallate, imipramine, serotonin, nicotine, cotinine, propylpyrazoletriol, genistein, pyrazole, ITE (2-(1H-Indol-3-ylcarbonyl)-4-thiazolecarboxylic acid methyl ester), MeBIO ((2′Z,3′E)-6-bromo-1-methylindirubin-3′-oxime), pifithrin-α, and any combination thereof.

12. The method of claim 1, wherein the method does not involve administering an estrogen receptor agonist.

13. The method according to claim 1, wherein the age-related condition or disorder is selected from cancer, neurodegenerative diseases, diabetes, obesity, or any combination thereof.

14. A dietary supplement comprising a compound selected from (i) an agonist of nuclear receptors that activate xenobiotic metabolism, (ii) a cardiac glycoside, (iii) a chelator, (iv) inulin, (v) D-valine, or any combination thereof.

15. The dietary supplement of claim 14, wherein the agonist of nuclear receptors that activate xenobiotic metabolism is selected from the group consisting of a CAR agonist, a PXR agonist, a PPARα agonist, and any combination thereof.

16. The dietary supplement of claim 15, wherein the CAR agonist is selected from the group consisting of 6,7-dimethylesculetin, acetaminophen, artemisinin, atorvastatin, cerivastatin, CITCO ((6-(4-chlorophenyl)imidazo[2,1-b][1,3]thiazole-5-carbaldehyde-O-(3,4-dichlorobenzyl)oxime), fluvastatin, orphenadrine, phenobarbital, phenytoin, pravastatin, simvastatin, and any combination thereof.

17. The dietary supplement of claim 15, wherein the PXR agonist is selected from 4-hydroxytamoxifen, androstenol, artemisinin, avasimibe, BK8644, bosentan, bromopropylate, butamifos, carbamazepine, cis-guggulsterone, clotrimazole, desmethoxyyangonin, dexamethasone, dihydromethysticin, dymuron, efavirenz, esprocarb, ethion, etoposide, flucythrinate, forskolin, hyperforin (hypericum perforatum extract or St John's wort), indanofan, isofenphos, isradipine, kava extract, lithocholic acid, lovastatin, meclizine, methadone, metlachlor, mevastatin, mifepristone, nicardipine, nifedipine, paclitaxel, PCN (pregnenolone-16α-carbonitrile), phenobarbital, piperophos, pretilachlor, pyributicarb, rifampicin, ritonavir, spironolactone, SR 12813, tamoxifen, thenylchlor, topiramate, topotecan, trans-guggulsteone, triadimefon, ursodeoxycholic acid, and any combination thereof.

18. The dietary supplement of claim 15, wherein the PPARα agonist is selected from aleglitazar, arachidonic acid, bezafibrate, ciprofibrate, clinofibrate, clofibrate, clofibric acid, clofibride, CP 775146, eicosapentaenoic acid, etofibrate, fenofibrate, fenofibric acid, gemfibrozil, GW 7647, linoleic acid, muraglitazar, nafenopin, oleic acid, oleylethanolamide, palmitic acid, palmitoleic acid, palmitoylethanolamide, pioglitazone, pirinixic acid, rivoglitazone, ronifibrate, rosiglitazone, saroglitazar, simfibrate, stearic acid, tesaglitazar, troglitazone, WY-14643, and any combination thereof.

19. The dietary supplement of claim 14, wherein the agonist of nuclear receptors that activate xenobiotic metabolism is selected from 2-naphthoflavone, 3-methylcholanthrene, dioxin, metyrapone, decitabine, trichostatin A, hydroxymethylpyrene, indolo[3,2-b]carbazole, phenethyl isothiocyanate, isothiocyanatomethylbenzene, sulforaphane, coumestrol, testosterone, dihydrotestosterone, indole-3-carbinol, 3-nitrobenzanthrone, 2-(4-amino-3-methylphenyl)-5-fluorobenzothiazole, primaquine, iprodione, ketoconazole, deltamethrin, omeprazole, pentachlorophenol, fipronil, sulindac, 3-aminobenzanthrone, 6-nitrochrysene, itraconazole, enilconazole (imazalil), 2-[2-(acetylamino)-4-(diallylamino)-5-methoxyphenyl]-5-amino-7-bromo-4-chloro-2H-benzotriazole (PTBA-8), 9-hydroxy-5,6-dimethyl-N-[2-(dimethylamino)ethyl)-6H-pyrido(4,3-b)-carbazole-1-carboxamide, carbaryl, 6-formylindolo[3,2-b]carbazole, phthalate, 2-[2-(acetylamino)-4-(diethylamino)-5-methoxyphenyl]-5-amino-7-bromo-4-chloro-2H-benzotriazole (PBTA-7), chlorpyrifos, sulindac sulfone, fluconazole, permethrin, ascorbigen, DEET (N,N-diethyl-meta-toluamide), clevidipine, emodin, 4-biphenylamine, nevirapine, efavirenz, chlorpyrifos oxon, ibrolipim, isoniazid, ethanol, tretinoin, fandosentan potassium, modafinil, trazodone, trimeprazine, etoposide, doxorubicin, rifabutin, alpha-naphthoflavone, progesterone, ethambutol, benzil, 1-methylphenanthrene, olopatadine, ethionamide, quercetin, 4-hydroxynonenal, oltipraz, butanoate, resveratrol, paraquat, alpha lipoic acid, carnosic acid, carnosol, oxaliplatin, paclitaxel, nicotinamide, azathioprine, eugenol, chlorophyllin, 2-tert-butylhydroquinone, hydrocortisone, procainamide, corticosterone, medroxyprogesterone 17-acetate, dopamine, 4-nitrophenol, bilirubin, glycyrrhizic acid, naringenin, saccharolactone, isopropyl thiogalactoside, naphthyl glucuronide, epigallocatechin gallate, imipramine, serotonin, nicotine, cotinine, propylpyrazoletriol, genistein, pyrazole, ITE (2-(1H-Indol-3-ylcarbonyl)-4-thiazolecarboxylic acid methyl ester), MeBIO ((2′Z,3′E)-6-bromo-1-methylindirubin-3′-oxime), pifithrin-α, and any combination thereof.

20. The dietary supplement of claim 14, wherein the cardiac glucoside is selected from the group consisting of acetyldigitoxin, acetyldigoxin, arenobufagin, bufotalin, cinobufagin, cymarin, deslanoside, Digitalis leaves, digitoxin, digoxin, gitoformate, k-strophanthin, lanatoside C, marinobufagin, metildigoxin, ouabain, peruvoside, proscillaridin, scilliroside, and any combination thereof.

21-23. (canceled)

24. A method of screening for a compound effective for treating a target condition or disorder in a mammal, the method comprising:
a) selecting a target condition or disorder,
b) selecting one hundred or more test compounds from each of twenty or more structural and/or functional classes of compounds, wherein (i) each of the test compounds has a known pharmacological or physiological effect or drug-like properties, and (ii) none of the selected test compounds are known, at the time of screening, to treat the target condition or disorder, or to substantially effect a biological pathway known to treat the target condition or disorder,
c) selecting a single route of administration and a single dosage form for the test compounds,
d) determining dosing amounts for each test compound based on (i) the selected route of administration, and (ii) known toxicity data,
e) optionally,
i) selecting one or more reference compounds for evaluation, wherein the reference compounds are known to treat the target condition or disorder, or to substantially effect a biological pathway known to treat the target condition or disorder,
ii) determining dosing amounts for each reference compound based on (i) the selected route of administration, (ii) known toxicity data, and optionally (iii) known efficacy data for the target condition or disorder,
f) performing high-throughput screening with the test compounds in mammals, the screening comprising for each test compound and reference compound,
i) administering the compound to a sufficient number of mammals having the condition or disorder or which is a model for the condition or disorder, such that a statistically significant number of mammals are administered the compound, and
ii) evaluating the target condition or disorder in mammal, and
g) selecting one or more of the evaluated test compounds which had positive evaluations for the target condition or disorder.

25-61. (canceled)
